(19) United States
(12) Patent Application Publication
(10) Pub. No.: US 2016/0143788 A1
(43) Pub. Date: May 26, 2016

(54) Ankle Blister Prevention Sleeve

(71) Applicant: Susan Hirsch, San Diego, CA (US)

(72) Inventor: Susan Hirsch, San Diego, CA (US)

(73) Assignee: HIRKOS INC, San Diego, CA (US)

(21) Appl. No.: 14/918,811

(22) Filed: Oct. 21, 2015

Related U.S. Application Data
(60) Provisional application No. 62/083,156, filed on Nov. 21, 2014.

(51) Int. Cl. A61F 13/06; A61F 13/00

(52) U.S. Cl. CPC A61F 13/066; A61F 13/00038; A61F 2013/00093; A61F 2013/00272

(57) ABSTRACT

In accordance with one embodiment an ankle blister prevention sleeve comprises a sleeve of elastic material with a contour to reduce the pressure applied to the calcaneus protrusion of the heel of the foot. The ankle blister prevention sleeve provides a means of protecting the skin of the foot from repetitive abrasion and subsequent blisters.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND

1. Field

This application relates to garments for the foot, more particularly protective sleeves for blister prevention.

2. Prior Art

The following is a list of some prior art that presently appears relevant:

U.S. PATENTS

| Patent Number | Issue Date | Name of Patentee |
|---|---|---|
| 4,296,499 | 1981 Oct. 27 | Patterson |
| 4,084,586 | 1978 Apr. 18 | Hettick |
| 6,059,744 | 2000 May 9 | Hardt |

U.S. PATENT APPLICATIONS

| Publication Number | Publication Date | Name of Applicant |
|---|---|---|
| 20070192938 | 2007 Aug. 23 | Yoon |

NON-PATENT LITERATURE DOCUMENTS

Valeo ® NAS Pull-On Ankle Support
http://www.valeowork.com/?id=10&prodId=164&CatId=45&Parent=35
and http://www.airgas.com/p/V14NAS-S/M retrieved 2014 Oct. 28
eZeeFit ® Ankle Bootie
http://www.ezeefitsports.com/category-s/1827.htm retrieved 2014 Nov. 4

One of the functions of footwear is to protect the skin of the foot from repetitive abrasion and subsequent blisters while not creating pressure points that can lead to bruising of the foot. The ankle and heel regions of the foot are particularly prone to blisters. The protrusion on the heel of the foot where the Achilles tendon meets the calcaneus bone is susceptible to bruising from excessive pressure from footwear.

Socks protect from blisters to some degree but are prone to wrinkle or slide on the foot and rub against the skin. Alternative foot coverings have been developed to provide improved protection from blisters. U.S. Pat. No. 4,296,499 to Patterson discloses a foot covering worn between the foot and sock which provides an additional slip layer between the sock and foot to reduce rubbing but some rubbing between the covering and the foot persists. U.S. patent application publication 20070192938 to Yoon discloses a tubular heel protector to be worn as a sock or under a sock. The disclosure of Yoon includes an inner pad coupled to the inside of the protector, said pad being intended to slide against the protector rather than against the foot to reduce rubbing on the foot. Yoon's protector does not provide relief of pressure on the calcaneus protrusion, and the sliding inner pad increases manufacturing complexity and will not serve its intended function if the static frictional coefficient between the pad and foot does not exceed the static friction coefficient between the pad and protector body.

U.S. Pat. No. 4,084,586 to Hettick discloses a tubular support made of an elastic material with an opening that leaves the heel exposed and prone to blisters. U.S. Pat. No. 6,059,744 to Hardt discloses and ankle support that does not provide relief of pressure on the calcaneus protrusion of the heel.

The Valeo® NAS Pull-On Ankle Support covers some of the foot but leaves the upper heel and Achilles tendon exposed and prone to blisters. The eZeeFit® Ankle Bootie covers the Achilles tendon and upper heel but applies pressure to the calcaneus protrusion of the heel.

From consideration of the foregoing there remains a need to provide a means of protecting the skin of the foot from repetitive abrasion and subsequent blisters while not creating pressure points that can lead to bruising of the foot.

### SUMMARY

In accordance with one embodiment an ankle blister prevention sleeve comprises a sleeve of elastic material with a contour to reduce the pressure applied to the calcaneus protrusion of the heel of the foot.

### DRAWINGS

### Figures

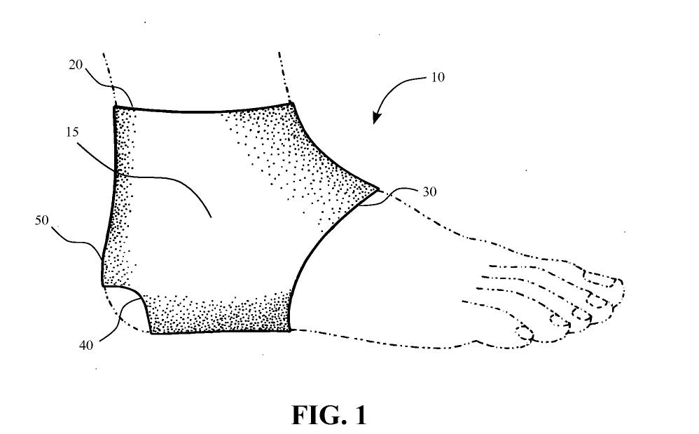

FIG. 1 shows an isometric view of an ankle blister protection sleeve on a foot in accordance with one embodiment. The phantom lines show the environmental structure of the foot on which the ankle protection sleeve is worn.

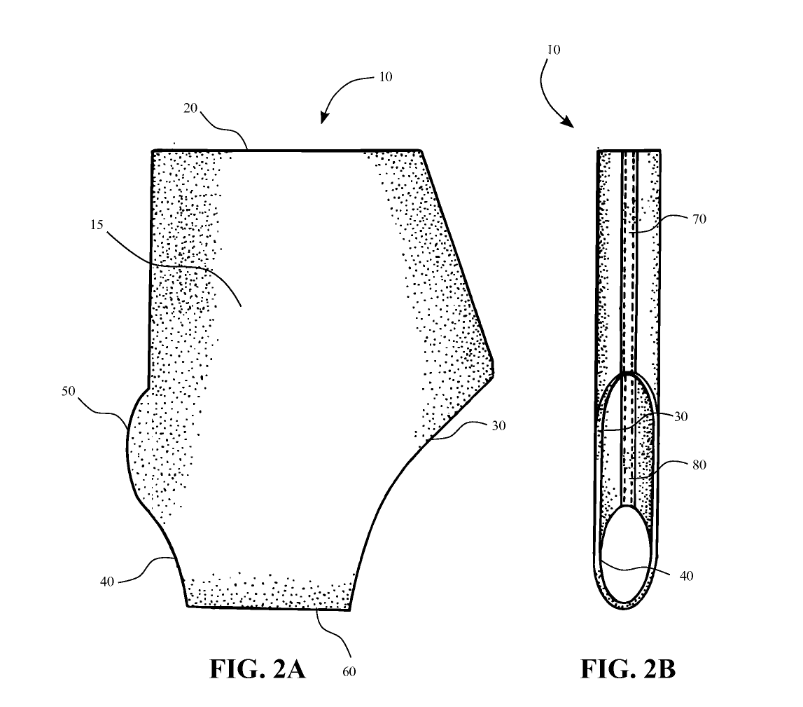

FIG. 2A and FIG. 2B show side and front views of an ankle blister protection sleeve in accordance with one embodiment.

### DETAILED DESCRIPTION

It is to be understood that the scope of the embodiments is not limited to the particular methods, compositions, and materials which are disclosed herein because such may vary. It should also be understood that all terminology used herein is only for the purpose of describing particular embodiments. These terms are not intended to be limiting in any way and should not be misconstrued to limit scope.

One embodiment of the ankle blister protection sleeve is show in FIG. 1 (isometric view on foot), FIG. 2A (side view), and FIG. 2B (front view), and is generally designated by the numeral 10. The sleeve is made from an elastic material 15. In accordance with one embodiment the elastic material 15 is spandex. In another embodiment the elastic material 15 contains a polyurethane core. The elastic material 15 also provides sufficient compression to prevent the sleeve from slipping relative to the foot. Such slipping could cause blisters of the foot.

The upper opening 20 provides for insertion of the foot into the sleeve. The forward opening 30 allows the front of the foot to be exposed. The heel opening 40 stabilizes the sleeve relative to the heel and avoids issues with bunched material at the heel of the sleeve. Both the forward opening 30 and the heel opening 40 are shown in the embodiment of FIG. 1, FIG. 2A, and FIG. 2B but one or both of these openings may be omitted in alternative embodiments.

A contour 50 in the sleeve accommodates the protrusion on the heel of the foot where the Achilles tendon meets the calcaneus bone. The contour 50 improves the fit of the sleeve by approximating the shape of the foot. The contour 50 reduces the pressure applied to the protrusion by the elastic material 15 and thus reduces the risk of bruising from excessive pressure. The contour 50 covers the protrusion on the heel of the foot where the Achilles tendon meets the calcaneus bone thereby protecting said protrusion from abrasion and preventing blisters.

In one embodiment the sleeve 10 is formed from a single piece of elastic material 15 which includes material to form the contour 50. The elastic material 15 is folded in half along the bottom 60. The elastic material 15 is then stitched together in front 70 and in back 80.

In another embodiment the sleeve 10 is formed by heat bonding the elastic material 15.

In another embodiment the sleeve 10 is formed by adhesive bonding the elastic material 15.

In another embodiment the sleeve 10 is formed from two or more pieces of elastic material 15.

In another embodiment the contour 50 is formed by heat-setting the elastic material 15 in the shape of the contour.

The ankle blister prevention sleeve may be worn directly on the foot. It may also be worn or over a sock, hosiery, support, or other foot covering including another ankle blister prevention sleeve. A sock, hosiery, support, or other covering may be worn over the ankle protection sleeve. Footwear including but not limited to shoes, boots, sandals, skates, fins, clogs, athletic equipment, toe shoes, etc. may be worn directly or indirectly over the ankle blister prevention sleeve.

It is to be understood that the aforementioned description and embodiments are merely exemplary and not scope limitations.

I claim:

1. An ankle garment comprising
a. an elastic material having an opening for receiving the foot, and
b. a contour in the heel region of said garment to accommodate the protrusion of the foot where the Achilles tendon meets the calcaneus bone
whereby said garment protects the skin from blisters.

2. The garment of claim 1 wherein said elastic material is spandex.

3. The garment of claim 1 wherein said elastic material includes a polyurethane core.

4. The garment of claim 1 wherein said garment is formed from said material by stitching.

5. The garment of claim 1 wherein said garment is formed from said material by heat bonding.

6. The garment of claim 1 wherein said garment is formed from said material by adhesive bonding.

7. The garment of claim 1 wherein said garment is formed from two or more pieces of said elastic material

8. The garment of claim 1 wherein said garment is formed by heat-setting said elastic material in the shape of said contour.
